(19) United States
(12) Patent Application Publication
(10) Pub. No.: US 2024/0238593 A1
(43) Pub. Date: Jul. 18, 2024

(54) METHODS AND SYSTEMS FOR DISEASE TREATMENT USING ELECTRICAL STIMULATION

(71) Applicant: Nevro Corp., Redwood City, CA (US)

(72) Inventors: Gary Heit, Woodside, CA (US); Bradford Evan Gliner, Sammamish, WA (US)

(21) Appl. No.: 18/622,434

(22) Filed: Mar. 29, 2024

Related U.S. Application Data
(63) Continuation of application No. 16/922,304, filed on Jul. 7, 2020, now Pat. No. 11,998,744.
Application No. 16/922,304 is a continuation of application No. 15/870,052, filed on Jan. 12, 2018, now Pat. No. 10,751,536.
Application No. 15/870,052 is a continuation of application No. 14/300,193, filed on Jun. 9, 2014, now Pat. No. 9,895,539.
(60) Provisional application No. 61/833,392, filed on Jun. 10, 2013.

(51) Int. Cl. A61N 1/36

(52) U.S. Cl. CPC A61N 1/36071; A61N 1/36062; A61N 1/36157; A61N 1/36171; A61N 1/36175

(57) ABSTRACT

Systems for disease treatment using electrical stimulation are disclosed. A representative method for treating a patient includes changing an activity, expression, or both activity and expression of a fast sodium channel, a glial cell, or both a fast sodium channel and a glial cell of the patient by applying to a target neural population of the patient an electrical therapy signal having a frequency in a frequency range of 1.5 kHz to 100 KHz.

### CROSS-REFERENCE TO RELATED APPLICATION

The present application is a continuation of U.S. patent application Ser. No. 14/300,193, filed Jun. 9, 2014, now issued as U.S. Pat. No. 9,895,539, which claims priority to U.S. Provisional Application 61/833,392, filed on Jun. 10, 2013, which are incorporated herein by reference.

### TECHNICAL FIELD

The present disclosure is directed generally to methods and systems for disease treatment using electrical stimulation. Particular embodiments include changing an activity, expression, or both activity and expression of a fast sodium channel, a glial cell, or both a fast sodium channel and a glial cell of the patient by applying electrical stimulation to a target neural population of a patient.

### BACKGROUND

Neurological stimulators have been developed to treat pain, movement disorders, functional disorders, spasticity, cancer, cardiac disorders, and various other medical conditions. Implantable neurological stimulation systems generally have an implantable signal generator and one or more leads that deliver electrical pulses to neurological tissue or muscle tissue. For example, several neurological stimulation systems for spinal cord stimulation (SCS) have cylindrical leads that include a lead body with a circular cross-sectional shape and one or more conductive rings (i.e., contacts) spaced apart from each other at the distal end of the lead body. The conductive rings operate as individual electrodes and, in many cases, the SCS leads are implanted percutaneously through a needle inserted into the epidural space, with or without the assistance of a stylet.

Once implanted, the signal generator applies electrical pulses to the electrodes, which in turn modify the function of the patient's nervous system, such as by altering the patient's responsiveness to sensory stimuli and/or altering the patient's motor-circuit output. In SCS therapy for the treatment of pain, the signal generator applies electrical pulses to the spinal cord via the electrodes. In conventional SCS therapy, electrical pulses are used to generate sensations (known as paresthesia) that mask or otherwise alter the patient's sensation of pain. For example, in many cases, patients report paresthesia as a tingling sensation that is perceived as less uncomfortable than the underlying pain sensation.

Aspects of the present disclosure are directed to systems and methods that make use of, employ, rely on and/or otherwise use or incorporate aspects the interaction between electrical therapy and the patients to whom the therapy is applied.

### BRIEF DESCRIPTION OF THE DRAWINGS

FIG. 1 is a partially schematic illustration of an implantable spinal cord modulation system positioned at the spine to deliver therapeutic signals in accordance with several embodiments of the present technology.

### DETAILED DESCRIPTION

Neurological stimulators have been developed to treat pain, movement disorders, functional disorders, spasticity, cancer, cardiac disorders, and various other medical conditions. Implantable neurological stimulation systems generally have an implantable pulse generator and one or more leads that deliver electrical pulses to neurological tissue or muscle tissue. For example, several neurological stimulation systems for spinal cord stimulation (SCS) have cylindrical leads that include a lead body with a circular cross-sectional shape and one or more conductive rings spaced apart from each other at the distal end of the lead body. The conductive rings operate as individual electrodes and, in many cases, the SCS leads are implanted percutaneously through a large needle inserted into the epidural space, with or without the assistance of a stylet.

Once implanted, the pulse generator applies electrical pulses to the electrodes, which in turn modify the function of the patient's nervous system, such as by altering the patient's responsiveness to sensory stimuli, altering the patient's motor-circuit output, and/or otherwise modifying other neural function. Example neuromodulation systems, methods, and therapy parameters are described in co-owned published patent applications: US Patent Publication No. 2009/0204173 (Atty. Dkt. No. 662458001US1); US Patent Publication No. 2007/0213771 (Atty. Dkt. No. 662458002US); US Patent Publication No. 2010/0191307 (Atty. Dkt. No. 662458011US); US Patent Publication No. 2010/0274312 (Atty. Dkt. No. 662458018US1); US Patent Publication No. 2010/0274314 (Atty. Dkt. No. 662458020US1); US Patent Publication No. 2012/0172946 (Atty. Dkt. No. 662458028US1); and US Patent Publication No. 2013/0066411 (Atty. Dkt. No. 662458033US3), which are all incorporated herein by reference in their entireties. To the extent the foregoing materials and/or any other materials incorporated herein by reference conflict with the present disclosure, the present disclosure controls.

Provided herein are various embodiments of neuromodulation systems, methods, and therapies for the treatment of medical conditions. The specific embodiments discussed are not to be construed as limitations on the scope of the disclosed technology. It will be apparent to one skilled in the art that various equivalents, changes, and modifications may be made without departing from the scope of the disclosed technology, and it is understood that such equivalent embodiments are to be included herein.

The following abbreviations are used herein: AIC, anterior limb internal capsule; BST, bed nucleus of the stria terminals; CMPF, centromedian and parafascicularis; CNS, central nervous system; DREZ, dorsal root entry zone; GF, genitofemoral; GNI, glial neuronal cell interaction; GPI, globus pallidus internus; MCS, motor cortex stimulation; MD, movement disorder; MI, primary motor cortex; ONS, occipital nerve stimulation; NAcc, nucleus accumbens; NTS, nucleus tractus solitarii; PVG, periventricular grey matter; PAG, periaqueductal grey matter; PPN, pedunculopontine nucleus; SCA, superior cerebellar artery; SCS, spinal cord stimulation; SMA, supplementary motor area; SPG, sphenopalatine ganglion; STN, subthalamic nucleus; Vcpc, ventro caudalis parvocellularis; VIP, ventral intermedia nucleus; VOA, Ventralis oralis anterior; VOP, ventralis oralis posterior; VPL, ventral posterolateral nucleus; VPM, ventral posteromedial nucleus; WDR, wide dynamic range; ZI, zona incerta.

Recent animal studies have shown that application of electrical stimulation to the dorsal root entry zone (DREZ) at frequencies between 2 kHz and 100 kHz suppresses wide dynamic range (WDR) neuron response by 70% in response to noxious stimulation (Cuellar 2012). Inhibition of WDR firing was found to persist for seconds to minutes after stimulation ended. WDR neurons (also known as convergent neurons) are one of three types of second order projection neurons. WDR neuron firing is correlated with pain perception, with firing rate increasing steadily as stimulus intensity increases. Thus, these data suggest that electrical stimulation at the tested frequencies functions in part by direct axonal inhibition.

Glial cells were traditionally thought to play primarily a structural role in the nervous system, for example by surrounding neurons, holding neurons in place, providing electrical insulation, and destroying pathogens. However, glial cells may play a role in the transmission of chronic pain by releasing various mediators such as nitric oxide, proinflammatory cytokines, excitatory amino acids, and prostaglandins. Release of these mediators may cause the release of substance P and excitatory amino acids by peripheral nerves as well as modify local neural interactions in the CNS, which in turn results in action potential generation or neural responses to synaptic inputs. Substance P and excitatory amino acid release can also further activate glial cells, creating a positive feedback loop. Glial cells form a network with themselves and communicate via slow inward calcium currents, which are activated by a variety of factors including potassium. Electrical stimulation with appropriate signal parameters may be used to reduce extracellular potassium levels by primary afferent inhibition, thereby reducing glial cell activity.

Neurons and certain glial cells contain sodium channels that are responsible for the rising phase of action potentials. When exposed to low frequencies, all of these sodium channels exhibit changes in their conductance. At higher frequencies, however, these changes are specific to fast sodium channels such as NaV1.8 and NaV1.9, which are overly active in chronic pain. Without being bound to a particular theory, electrical stimulation with appropriate signal parameters may derive pain reduction in part from its ability to change the conductance of fast sodium channels in neurons and/or glial cells, thereby specifically downregulating those sodium channels that are most involved with chronic pain.

As disclosed herein, electrical stimulation, with the therapy signal parameters disclosed herein, can be used to normalize pathological neural networks associated with fast sodium channel activity and/or expression by attenuating pathology-induced sodium channel activity and modulating glial neuronal cell interaction (GNI). GNI accordingly refers generally to interactions with a glial/neuronal component, including interactions between (a) glial cells and other glial cells, (b) glial cells and neurons, (c) glial networks and neurons, and/or (d) glial networks and neural networks. Based on this, the present application provides methods and devices for attenuating pathology-induced sodium channel activity, (and/or other pathology-induced ionophores or membrane channel activity) modulating GNI, and treating various conditions associated with fast sodium channel activity and/or expression and GNI.

In certain embodiments, methods are provided for attenuating pathology-induced sodium channel activity by applying electrical stimulation, with the therapy signal parameters disclosed herein, to a target tissue or organ. This attenuation may result in decreased activity and/or expression of one or more fast sodium channels, including for example NaV1.8 or NaV1.9. In certain embodiments, decreased activity and/or expression of one or more fast sodium channels results in decreased glial cell and/or neuronal activity. In certain embodiments, attenuation of pathology-induced sodium channel activity may also result in increased activity and/or expression of one or more slow sodium channels, including for example NaV1.3.

In certain embodiments, methods are provided for modulating GNI by applying electrical stimulation, with the therapy signal parameters disclosed herein, to a target tissue or organ. In certain embodiments, this modulation may result in a decrease in the release of one or more mediators by glial cells, including for example nitric oxide, proinflammatory cytokines, excitatory amino acids, and prostaglandins. In certain embodiments, this decrease may result in a decrease in action potential generation by one or more peripheral nerves or neural elements in CNS networks, and in certain of these embodiments, the decrease may result in reduction or cessation of one or more symptoms of a medical condition (e.g., chronic pain).

In certain embodiments, methods are provided for treating a condition associated with fast sodium channel activity and/or expression, or a condition for which attenuated fast sodium channel activity and/or expression is expected to be beneficial, by applying electrical stimulation, with the therapy signal parameters disclosed herein, to a target tissue or organ. In certain embodiments, the condition being treated is selected from the group consisting of a chronic pain condition, a movement disorder, dysautonomia, an anxiety disorder, a cognitive disorder, a development disorder, a metabolic disease, or a mood disorder.

In certain embodiments, methods are provided for treating a chronic pain condition by applying electrical stimulation, with the therapy signal parameters disclosed herein, to a target tissue or organ. In certain of these embodiments the chronic pain condition is a headache pain syndrome, fascial pain syndrome, neck and brachial plexus pain syndrome, shoulder pain syndrome, elbow pain syndrome, other upper extremity pain syndrome, wrist pain syndrome, hand pain syndrome, chest wall pain syndrome, thoracic spine pain syndrome, abdominal & groin pain syndrome, lumbar spine & sacroiliac joint pain syndrome, pelvic pain syndrome, hip & lower extremity pain syndrome, knee pain syndrome, ankle pain syndrome, foot pain syndrome, visceral pain or whole body pain syndromes. In certain of these embodiments, the chronic pain disorder may be a condition listed in Table 1. Table 1 provides various spinal cord, cortical, sub-cortical, and/or peripheral targets for applying electrical stimulation in the treatment of each condition. Treatment may be carried out by applying electrical stimulation to any of the targets listed, or to a combination thereof. The list of targets is not exhaustive, meaning that there may be one or more additional targets for each condition.

TABLE 1
Chronic pain conditions

| | Spinal Cord | Cortical | Sub-Cortical | Peripheral |
|---|---|---|---|---|
| Indication | Target | Target | Target | Target |
| Headache Pain | C2-C4, (e.g., | Motor cortex | Periventricular | Sphenopalatine |
| Syndromes | C2-C3) | stimulation | grey matter | ganglion (SPG) |
| | | (MCS), | (PVG), | |
| | | Posterior | periaqueductal | |
| | | cingulum and | grey matter | |
| | | cingulate | (PAG), | |
| | | gyrus. | (nociceptive | |
| | | | pain); internal | |
| | | | capsule, ventral | |
| | | | posterolateral | |
| | | | nucleus (VPL), | |
| | | | ventral | |
| | | | posteromedial | |
| | | | nucleus (VPM) | |
| | | | (neuropathic | |
| | | | pain) | |
| Herpes Zoster | Appropriate | Posterior | Ventro caudalis | Gasserian |
| (shingles) - | spinal level | cingulum and | parvocellularis | ganglion, |
| Trigeminal | | cingulate | (Vcpc), | Sphenopalatine |
| | | gyrus. | thalamus, NAcc | ganglion (SPG) |
| Migraine | C1-C2 | Posterior | Hypothalamus | Sphenopalatine |
| | | cingulum and | | ganglion (SPG), |
| | | cingulate | | Gasserian |
| | | gyrus. | | ganglion, |
| | | | | occipital nerve |
| | | | | stimulation (ONS) |
| Cluster | | | Hypothalamus | SPG, Gasserian |
| | | | | ganglion |
| Analgesic Rebound | | | PVG, PAG | |
| Occipital Neuralgia | C1-C2 | | PVG, PAG, | ONS |
| | | | Vcpc | |
| Fascial Pain | C2-C4, (e.g., | MCS, Posterior | Vcpc, thalamus | SPG, Gasserian |
| Syndromes | C2-C3) | cingulum and | | ganglion |
| | | cingulate | | |
| | | gyrus. | | |
| Trigeminal | | | Vcpc, thalamus | SPG, Gasserian |
| Neuralgia | | | | ganglion |
| Temporomandibular | | | Vcpc, thalamus | SPG, Gasserian |
| Joint Dysfunction | | | | ganglion, |
| | | | | superficial |
| | | | | temporal nerve |
| Trigeminal | | MCS, Posterior | Vcpc, thalamus, | SPG, Gasserian |
| Neuropathy (aka | | cingulum and | NAcc | ganglion, |
| Atypical Facial | | cingulate gyrus | | superficial |
| Pain) | | | | temporal nerve |
| Myofascial Pain | | Posterior | NAcc | SPG, Gasserian |
| Syndrome - Face | | cingulum and | | ganglion, |
| | | cingulate gyrus | | superficial |
| | | | | temporal nerve |
| Cancer Pain | | Insular cortex, | PVG, PAG, | SPG, Gasserian |
| | | Posterior | nucleus | ganglion, |
| | | cingulum and | accumbens | superficial |
| | | cingulate gyrus | (NAcc), Vcpc | temporal nerve |
| | | | Thalamus | |
| Hyoid Syndrome | C1-C3 | | | SPG, Gasserian |
| | | | | ganglion, |
| | | | | superficial |
| | | | | temporal nerve |
| Reflex Sympathetic | | Insular Cortex, | Vcpc, thalamus, | SPG, Gasserian |
| Dystrophy-Face | | Posterior | hypothalamus | ganglion |
| | | cingulum and | | |
| | | cingulate gyrus | | |
| Neck & Brachial | C2-C6, (e.g., | | | |
| Plexus Pain | C3-C4) | | | |
| Syndromes | | | | |
| Cervical Facet | Appropriate | | | |
| Syndrome | somatotopic | | | |
| | spinal level | | | |
| Cervical | Appropriate | | | |
| Radiculopathy | somatotopic | | | |
| | spinal level | | | |
| Fibromyalgia - | Appropriate | | | |
| Cervical | somatotopic | | | |
| Musculature | spinal level | | | |
| Myofascial Pain | Appropriate | | | |
| Syndrome - | somatotopic | | | |
| Cervical | spinal level | | | |
| Musculature | | | | |
| Brachial Plexopathy | C3-C8 | MCS, Posterior | Vcpc, thalamus, | |
| | (depending on | cingulum and | PAG, PVG, | |
| | site of pain) | cingulate gyrus | centromedian | |
| | | | and | |
| | | | parafascicularis | |
| | | | (CMPF), NAcc | |
| Pancoast | C3-C5 | Posterior | PVG, PAG, | |
| Syndrome | | cingulum and | pulvinar | |
| | | cingulate gyrus | | |
| Thoracic Outlet | C4 or C8 | | | |
| Syndrome | | | | |
| Shoulder Pain | C2-C6, (e.g., | MCS, , | Vcpc thalamus, | Brachial plexus |
| Syndromes | C3-C5) | Posterior | PAG, PVG, | |
| | | cingulum and | CMPF, NAcc | |
| | | cingulate gyrus | | |
| Arthritis Pain - | C3-C5 | MCS, Posterior | Vcpc thalamus, | Brachial plexus |
| Shoulder | | cingulum and | PAG, PVG, | |
| | | cingulate gyrus | CMPF, NAcc | |
| Acromioclavicular | C4 | | | Brachial plexus |
| Joint Pain | | | | |
| Myofascial Pain | C4 | | | Brachial plexus |
| Syndrome - | | | | |
| Shoulders | | | | |
| Subdeltoid Bursitis | C4 | | | Brachial plexus |
| Bicipital Tendonitis | C4 | | | Brachial plexus |
| Supraspinatus | C2-C4 | | | Brachial plexus |
| Syndrome | | | | |
| Rotator Cuff Tear | C3-C5 (e.g., | | | Brachial plexus |
| | post-surgery) | | | |
| Deltoid Syndrome | C-C5 | | | Brachial plexus |
| Teres Major | C3-C6 | | | Brachial plexus |
| Syndrome | | | | |
| Scapulocostal | C3-C7 | | | Brachial plexus |
| Syndrome | | | | |
| Elbow Pain | C2-C6, (e.g., | | | Brachial plexus/ |
| Syndromes | C3-C5) | | | ulnar nerve |
| Arthritis Pain-Elbow | C4-C8 | | | |
| Tennis Elbow | C6-C8 | | | |
| Golfer's Elbow | C6-C8 | | | |
| Anconeus | C6-C8 | | | |
| Compartment | | | | |
| Syndrome | | | | |
| Supinator | C6-C8 | | | |
| Syndrome | | | | |
| Brachioradialis | C6-C8 | | | |
| Syndrome | | | | |
| Ulnar Nerve | C6-C8 | | | |
| Entrapment At The | | | | |
| Elbow | | | | |
| Lateral Antebrachial | C6-C8 | | | |
| Cutaneous Nerve | | | | |
| Syndrome | | | | |
| Olecranon Bursitis | C6-C8 | | | |
| Other Upper | C2-C6, (e.g., | | | Brachial plexus |
| Extremity Pain | C3-C5) | | | |
| Syndromes | | | | |
| Phantom Limb Pain | C2-C8, L1-S1 | MCS, post- | Vcpc, cingulum, | |
| | | cingulum, | NAcc | |
| | | insula | | |
| Wrist Pain | C2-C6, (e.g., | | | |
| Syndromes | C3-C5) | | | |
| Arthritis Pain - Wrist | C6-C8 | | | |
| Carpal Tunnel | C6-C8 (e.g., | | | |
| Syndrome | post-surgery) | | | |
| De Quervain's | C5-C6 | | | |
| Tenosynovitis | | | | |
| Arthritis Pain - | C6-T1 | | | |
| Carpometacarpal | | | | |
| Joints | | | | |
| Hand Pain | C2-C6, (e.g., | | | |
| Syndromes | C3-C5) | | | |
| Arthritis Pain - | C5-T1 | | | |
| Fingers | | | | |
| Trigger Thumb | C5-C7 | | | |
| Trigger Finger | C5-C7 | | | |
| Ganglion Cysts of | C5-C7 | | | |
| Wrist & Hand | | | | |
| Sesamoiditis of the | C5-C7 | | | |
| Hand | | | | |
| Chest Wall Pain | T1-T12 | | | |
| Syndromes | | | | |
| Intercostal | Level of | | | Costal Nerve |
| Neuralgia | neuralgia | | | |
| Post-Thoracotomy | Level of | | | |
| Pain | surgery +/−1 | | | |
| | level | | | |
| Thoracic Spine | T1-T12 | | | |
| Pain Syndromes | | | | |
| Cancer Pain | Level of pain | Insular cortex, | PVG, PAG, | |
| | +2 levels | post cingulate | cingulum, NAcc | |
| | | cortex | | |
| Costovertebral | Center on | | | |
| Arthritis Pain | dermatome | | | |
| Postherpetic | Center on | | NAcc, Vc | Center on |
| Neuralgia | dermatome | | Thalmus | dermatome |
| Abdominal & | T6-T12 | | | Left vagus nerve, |
| Groin Pain | | | | subdiaphragmatic |
| Syndromes | | | | right vagus nerve |
| Cancer Pain | | Insular cortex, | PVG, PAG, | |
| | | posterior | NAcc, cingulum | |
| | | cingulate | | |
| | | cortex | | |
| Chronic Pancreatitis | | Insular cortex, | | Left vagus nerve, |
| | | posterior | | subdiaphragmatic |
| | | cingulate | | right vagus nerve |
| | | cortex | | |
| Ilioinguinal | | | | Ilioinguinal nerve |
| Neuralgia | | | | (field app) |
| Visceral Pain | | Insular cortex, | PVG, PAG, | Left vagus |
| (peritoneum, | | posterior | NAcc, cingulum | |
| stomach, | | cingulate | | |
| duodenum, | | cortex | | |
| intestine, colon, | | | | |
| liver, spleen, | | | | |
| pancreas, kidney, | | | | |
| adrenal gland, | | | | |
| appendix, gall | | | | |
| bladder) | | | | |
| Post-vasectomy | | | | Genitofemoral |
| Pain Syndrome | | | | (GF) nerve |
| Genitofemoral | | | | GF nerve |
| Neuralgia | | | | |
| Lumbar Spine & | T8-T12 | Insular cortex, | PVG, PAG, | |
| Sacroiliac Joint | | post cingulate | NAcc, cingulum, | |
| Pain Syndromes | | cortex | Vcpc | |
| Myofascial Pain | T8-T12 | | | |
| Syndrome | | | | |
| Lumbar | Segment | | | |
| Radiculopathy | appropriate +/− | | | |
| | 2 levels | | | |
| Latissimus Dorsi | Upper thoracic | | | |
| Muscle Syndrome | | | | |
| Arachnoiditis | S1-L3 (tune to | Insular cortex, | PVG, PAG | |
| | area of pain) | posterior | NAcc, cingulum | |
| | | cingulate | | |
| | | cortex | | |
| Sacroiliac Joint | S1 and L1 | Insular cortex, | | |
| Pain | | post cingulate | | |
| | | cortex | | |
| Pelvic Pain | T12-L5 | | | |
| Syndromes | | | | |
| Cancer Pain | NO | Insular cortex, | PVG, PAG, | |
| | | post cingulate | NAcc, cingulum | |
| | | cortex | | |
| Gluteus Maximus | | | | |
| Syndrome | | | | |
| Visceral Pain | | Insular cortex, | PVG, PAG, | Pudendal nerve |
| (pelvis, coccyx, | | posterior | NAcc, PO | |
| ovaries, fallopian | | cingulate | thalamus, | |
| tube, uterus, vulva, | | cortex | cingulum | |
| clitoris, perineum, | | | | |
| urinary bladder, | | | | |
| testicles, rectum) | | | | |
| Piriformis | | Insular cortex, | | |
| Syndrome | | post cingulate | | |
| | | cortex | | |
| Ischiogluteal | | | | Pudendal nerve |
| Bursitis | | | | |
| Levator Ani | S3-S4 | | | Pudendal nerve |
| Syndrome | | | | |
| Coccydynia | S3-S4 | | | Pudendal nerve |
| Hip & Lower | T8-T12 | | | |
| Extremity Pain | | | | |
| Syndromes | | | | |
| Arthritis Pain - Hip | T11-L3 | | | |
| Meralgia | L1 | | | |
| Paresthetica | | | | |
| Phantom Limb Pain | | MCS | Vcpc | |
| Knee Pain | T8-T12 | | | |
| Syndromes | | | | |
| Ankle Pain | T8-T12 | | | |
| Syndromes | | | | |
| Foot Pain | T8-T12 | | | |
| Syndromes | | | | |
| Arthritis - Toe Pain | T10-S1 | | | |
| Bunion Pain | T10-S1 | | | |
| Plantar Fasciitis | T11-L3 | | | |
| Calcaneal Spur | T11-L3 | | | |
| Syndrome | | | | |
| Whole Body Pain | C2-C4 | | | |
| Syndromes | | | | |
| Cancer Pain | | Insula cortex, | PVG, PAG, | |
| | | post cingulate | cingulum, | |
| | | cortex | posterior | |
| | | | thalamus | |
| Chronic Regional | C4-C8, T8-T12 | Insular cortex, | Hypothalamus, | |
| Pain Syndrome - | | post cingulate | posterior | |
| Multiple Limb | | cortex | thalamus | |
| Phantom pain | | Insular cortex, | Cingulum, PVG, | |
| syndromes | | posterior | PAG, Vcpc, | |
| | | cingulate | thalamus | |
| | | cortex, S1, S2 | | |
| | | cortex | | |

In certain embodiments, methods are provided for treating a movement disorder by applying electrical stimulation, with the therapy signal parameters disclosed herein, to a target tissue or organ. In certain of these embodiments, the movement disorder may be a condition listed in Table 2. Table 2 provides various spinal cord, cortical, sub-cortical, and/or peripheral targets for applying electrical stimulation in the treatment of each condition. Treatment may be carried out by applying electrical stimulation to any of the targets listed, or to a combination thereof. The list of targets is not exhaustive, meaning that there may be one or more additional targets for each condition.

TABLE 2
Movement disorders

| | Spinal | | | Periph- |
|---|---|---|---|---|
| | Cord | Cortical | Sub-Cortical | eral |
| Indication | Target | Target | Target | Target |
| Akathisia | | Primary motor | STN, Globus | |
| (inability | | cortex (MI), | pallidus internus | |
| to sit still) | | supplementary | (GPI), ventralis | |
| | | motor area | oralis anterior | |
| | | (SMA) | (VOA), ventralis | |
| | | | oralis posterior | |
| | | | (VOP), | |
| | | | subthalamic | |
| | | | nucleus (STN) | |
| Akinesia | | | STN, | |
| (lack of | | | Pedunculopontine | |
| movement) | | | nucleus (PPN), | |
| | | | mid-thalamic | |
| | | | intralaminar and | |
| | | | reticular nuclei | |
| Associated | | MI, SMA | GPI, VOA, VOP, | |
| Movements | | | STN, zona incerta | |
| (Mirror | | | (ZI), area Q | |
| Movements or | | | | |
| Homolateral | | | | |
| Synkinesis) | | | | |
| Athetosis | | MI, SMA | GPI, VOA, VOP, | |
| (contorted | | | STN | |
| torsion | | | | |
| or twisting) | | | | |
| Ataxia (gross | | MI, SMA | GPI, VOA, VOP, | |
| lack of | | | STN | |
| coordination | | | | |
| of muscle | | | | |
| movements) | | | | |
| Ballismus (violent | | MI, SMA | GPI, VOA, VOP, | |
| involuntary rapid | | | STN | |
| and irregular | | | | |
| movements) | | | | |
| Hemiballismus | | | GPi, VoA, VoP, | |
| (affecting only | | | STN | |
| one side | | | | |
| of the body) | | | | |
| Bradykinesia | | MI, SMA | GPI, VOA, VOP, | |
| (slow | | | STN | |
| movement) | | | | |
| Cerebral Palsy | | MI, SMA | Deep cerebellar | |
| | | | nuclei | |
| Chorea (rapid, | | MI, SMA | GPI, VOA, VOP, | |
| involuntary | | | STN | |
| movement) | | | | |
| Sydenham's | | MI, SMA | GPI, VOA, VOP, | |
| Chorea | | | STN | |
| Rheumatic | | MI, SMA | GPI, VOA, VOP, | |
| Chorea | | | STN | |
| Huntington's | | MI, SMA | GPI, VOA, VOP, | |
| Disease | | | STN | |
| Dystonia | | MI, SMA | GPI, VOA, VOP, | |
| (sustained | | | STN | |
| torsion) | | | | |
| Dystonia DTY1, | | MI, SMA | GPI, VOA, VOP, | |
| DTY11 and | | | STN | |
| generalized | | | | |
| dystonia | | | | |
| Blepharospasm | | MI, SMA | GPI, VOA, VOP | |
| | | | STN | |
| Writer's Cramp | | MI, SMA | GPI, VOA, VOP | |
| | | | STN | |
| Spasmodic | | MI, SMA | GPI, VOA, VOP | |
| Torticollis | | | STN | |
| (twisting of | | | | |
| head and neck) | | | | |
| Dopamine- | | MI, SMA | GPI, VOA, VOP | |
| Responsive | | | STN | |
| Dystonia | | | | |
| (hereditary | | | | |
| progressive | | | | |
| dystonia with | | | | |
| diurnal | | | | |
| fluctuation | | | | |
| or Segawa's | | | | |
| disease) | | | | |
| Geniospasm | | MI, SMA | GPI, VOA, VOP | |
| (episodic | | | STN | |
| involuntary | | | | |
| up and down | | | | |
| movements | | | | |
| of the chin and | | | | |
| lower lip) | | | | |
| Myoclonus | | MI, SMA | GPI, VOA, VOP | |
| (brief, | | | STN | |
| involuntary | | | | |
| twitching of a | | | | |
| muscle or | | | | |
| a group | | | | |
| of muscles) | | | | |
| Metabolic | | MI, SMA | GPI, VOA, VOP | |
| General | | | STN | |
| Unwellness | | | | |
| Movement | | | | |
| Syndrome | | | | |
| (MGUMS) | | | | |
| Parkinson's | | Motor cortex, | Subthalamic | |
| Disease | | pre-motor | nucleus, GPI, ZI, | |
| | | cortex | pallidofugal fibers | |
| Spasms | | | Superior | |
| (contractions) | | | cerebellar artery | |
| | | | (SCA), Superior | |
| | | | Cerebellar | |
| | | | structures, deep | |
| | | | cerebellar nuc | |
| Tardive | | | STN, GPI, VOA, | |
| dyskinesia | | | VOP | |
| Tic Disorders | | | Anterior limb | |
| (involuntary, | | | internal capsule | |
| compulsive, | | | (AIC), VOA, | |
| repetitive, | | | CMPF thalamus | |
| stereotyped) | | | | |
| Tourette's | | | AIC, GPI, VOA, | |
| Syndrome | | | VOP, STN, | |
| | | | CMPF | |
| Tremor | | | Ventral | |
| (oscillations) | | | intermedia | |
| | | | nucleus (VIM), | |
| | | | Area Q, ZI | |
| Rest Tremor | | | STN, GPI, Area | |
| (4-8 Hz) | | | Q, ZI | |
| Postural Tremor | | | STN, VIM, Area | |
| | | | Q, ZI | |
| Kinetic Tremor | | | VIM, Area Q, ZI | |
| Essential Tremor | | | VIM, Area Q, ZI | |
| (6-8 Hz variable | | | | |
| amplitude) | | | | |
| Cerebellar tremor | | | VIM, deep | |
| (6-8 Hz variable | | | cerebellar nuclei, | |
| amplitude) | | | Area Q | |
| Parkinsonian | | | STN +/− VIM | |
| tremors (4-8 Hz | | | | |
| variable | | | | |
| amplitude) | | | | |
| Physiological | | | VIM, Area Q, ZI | |
| tremor (10-12 Hz | | | | |
| low amplitude) | | | | |
| Wilson's disease | | | VIM and/or STN | |

In certain embodiments, methods are provided for treating a dysautonomic condition by applying electrical stimulation, with the therapy signal parameters disclosed herein, to a target tissue or organ. In certain of these embodiments, the dysautonomic condition may be a condition listed in Table 3. Table 3 provides various spinal cord, cortical, sub-cortical, and/or peripheral targets for applying electrical stimulation in the treatment of each condition. Treatment may be carried out by applying electrical stimulation to any of the targets listed, or to a combination thereof. The list of targets is not exhaustive, meaning that there may be one or more additional targets for each condition.

TABLE 3
Dysautonomic conditions

| | Spinal | | | |
|---|---|---|---|---|
| | Cord | Cortical | Sub-Cortical | Peripheral |
| Indication | Target | Target | Target | Target |
| Postural Orthostatic | T2-T5 | insula | Hypothalamus | |
| Tachycardia | | | | |
| Syndrome (POTS) | | | | |
| Inappropriate Sinus | T2-T5 | insula | Hypothalamus | |
| Tachycardia (IST) | | | | |
| Vasovagal Syncope | T2-T5 | insula | NTS | |
| Neurocardiogenic | | insula | Nucleus tractus | Right vagus |
| Syncope (NCS) | | | solitarii (NTS) | nerve, left |
| | | | | vagus nerve |
| Neurally Mediated | | insula | Hypothalamus | |
| Hypotension (NMH) | | | | |
| Autonomic Instability | T2-T5 | insula | | |

In certain embodiments, methods are provided for treating an anxiety disorder by applying electrical stimulation, with the therapy signal parameters disclosed herein, to a target tissue or organ. In certain of these embodiments, the anxiety disorder may be a condition listed in Table 4. Table 4 provides various spinal cord, cortical, intra-cortical, and/or peripheral targets for applying electrical stimulation in the treatment of each condition. Treatment may be carried out by applying electrical stimulation to any of the targets listed, or to a combination thereof. The list of targets is not exhaustive, meaning that there may be one or more additional targets for each condition.

TABLE 4
Anxiety disorders

| | Spinal | | | |
|---|---|---|---|---|
| | Cord | Cortical | Intra-Cortical | Peripheral |
| Indication | Target | Target | Target | Target |
| Generalized | | Parietal, | Amygdala, | |
| Anxiety | | prefrontal | insula, cingulate, | |
| Disorder | | | DM thalamus | |
| Phobic | | Insular cortex, | Corpus | |
| Disorder | | medial | callosum, | |
| | | prefrontal | hippocampus, | |
| | | cortex, anterior | ventral striatum, | |
| | | cingulate | bed nucleus of | |
| | | cortex, | the stria | |
| | | ventromedial | terminals (BST), | |
| | | prefrontal | amygdala, septal | |
| | | cortex | nuclei | |
| Specific | | Insular cortex, | Amygdala, | |
| Phobias (e.g., | | medial | NAcc, septal | |
| arachnophobia, | | prefrontal | nuclei | |
| acrophobia | | cortex, anterior | | |
| | | cingulate | | |
| | | cortex, | | |
| | | ventromedial | | |
| | | prefrontal | | |
| | | cortex | | |
| Social Phobias, | | Insular cortex, | Amygdala, | |
| (e.g., public | | medial | NAcc, septal | |
| speaking, | | prefrontal | nuclei | |
| crowded | | cortex, anterior | | |
| areas) | | cingulate | | |
| | | cortex, | | |
| | | ventromedial | | |
| | | prefrontal | | |
| | | cortex | | |
| Agoraphobia | | Insular cortex, | NAcc, BST, | |
| | | medial | amygdala | |
| | | prefrontal | | |
| | | cortex, anterior | | |
| | | cingulate | | |
| | | cortex, | | |
| | | ventromedial | | |
| | | prefrontal | | |
| | | cortex | | |
| Panic Disorder | | Insular cortex, | NAcc, BST, | |
| | | medial | ventral striatum, | |
| | | prefrontal | DM thalamus | |
| | | cortex, anterior | | |
| | | cingulate | | |
| | | cortex, | | |
| | | ventromedial | | |
| | | prefrontal | | |
| | | cortex | | |
| Obsessive | | Cg 25- | AIC, CMPF | |
| Compulsive | | cingulate | thalamus | |
| Disorder | | cortex, | | |
| (OCD) | | orbitofrontal | | |
| | | cortex | | |

In certain embodiments, methods are provided for treating a cognitive disorder by applying electrical stimulation, with the therapy signal parameters disclosed herein, to a target tissue or organ. In certain of these embodiments, the cognitive disorder may be a condition listed in Table 5. Table 5 provides various spinal cord, cortical, sub-cortical, and/or peripheral targets for applying electrical stimulation in the treatment of each condition. Treatment may be carried out by applying electrical stimulation to any of the targets listed, or to a combination thereof. The list of targets is not exhaustive, meaning that there may be one or more additional targets for each condition.

TABLE 5
Cognitive disorders

| | Spinal | | | |
|---|---|---|---|---|
| | Cord | Cortical | Sub-Cortical | Peripheral |
| Indication | Target | Target | Target | Target |
| Dementia | | Entorhinal | Precommissural | |
| | | cortex, | fornix | |
| | | hippocampus | | |
| Amnesia | | Entorhinal | Precommissural | |
| | | cortex, | fornix | |
| | | hippocampus | | |

In certain embodiments, methods are provided for treating a development disorder by applying electrical stimulation, with the therapy signal parameters disclosed herein, to a target tissue or organ. In certain of these embodiments, the development disorder may be a condition listed in Table 6. Table 6 provides various spinal cord, cortical, sub-cortical, and/or peripheral targets for applying electrical stimulation in the treatment of each condition. Treatment may be carried out by applying electrical stimulation to any of the targets listed, or to a combination thereof. The list of targets is not exhaustive, meaning that there may be one or more additional targets for each condition.

TABLE 6
Development disorders

| | Spinal | | | |
|---|---|---|---|---|
| | Cord | Cortical | Intra-cortical | Peripheral |
| Indication | Target | Target | Target | Target |
| Motor disorders | | | GPI, VOA, VOP, | |
| | | | deep cerebellar | |
| | | | nuclei, | |
| | | | Cerebellar | |
| | | | vermis | |

In certain embodiments, methods are provided for treating a metabolic disease by applying electrical stimulation, with the therapy signal parameters disclosed herein, to a target tissue or organ. In certain of these embodiments, the metabolic disease may be selected from the group consisting of diabetes mellitus, an acid-base imbalance, a metabolic brain disease, a calcium metabolism disorder, a DNA repair deficiency disorder, an inborn metabolic error disorder, a mitochondrial disease, or a porphyria, and in certain of these embodiments the metabolic disease may be a condition listed in Table 7. Table 7 provides various spinal cord, cortical, sub-cortical, and/or peripheral targets for applying electrical stimulation in the treatment of each condition. Treatment may be carried out by applying electrical stimulation to any of the targets listed, or to a combination thereof. The list of targets is not exhaustive, meaning that there may be one or more additional targets for each condition.

TABLE 7
Metabolic diseases

| | Spinal | | | |
|---|---|---|---|---|
| | Cord | Cortical | Sub-Cortical | Peripheral |
| Indication | Target | Target | Target | Target |
| Diabetes Mellitus | | | | |
| Type I Diabetes | | | Hypothalamus | Splenic |
| | | | | and |
| | | | | gastric |
| | | | | nerve |
| Type II Diabetes | | | Hypothalamus | Splenic |
| | | | | and |
| | | | | gastric |
| | | | | nerve |
| Acid-Base | | | Hypothalamus, | |
| Imbalance | | | subfornical | |
| | | | organ of pines | |
| Acidosis+ | | | Hypothalamus, | |
| | | | subfornical | |
| | | | organ of pines | |
| Alkalosis+ | | | Hypothalamus, | |
| | | | subfornical | |
| | | | organ of pines | |
| Brain Diseases, | | | | |
| Metabolic | | | | |
| Hepatic | | | GPI, VOA, VOP, | |
| Encephalopathy | | | thalamus | |
| (HE) | | | | |
| Kernicterus | | | GPI, VOA, VOP, | |
| | | | thalamus | |
| Mitochondrial | | | GPI, VOA, VOP, | |
| Encephalo- | | | thalamus | |
| myopathies | | | | |
| Wernicke | | Entorhinal | Fornix, | |
| Encephalopathy | | cortex | mammillary | |
| | | | bodies | |
| DNA Repair | | | | |
| Deficiency | | | | |
| Disorders | | | | |
| Ataxia | | | GPI, VOA, VOP, | |
| Telangiectasia | | | thalamus | |
| Bloom Syndrome | | | GPI, VOA, VOP, | |
| | | | thalamus | |
| Cockayne | | | GPI, VOA, VOP, | |
| Syndrome | | | thalamus | |
| Fanconi Anemia | | | GPI, VOA, VOP, | |
| | | | thalamus | |
| Metabolism, | | | GPI, VOA, VOP (to | |
| Inborn | | | the extent subjects | |
| Errors | | | have movement | |
| | | | disorders (MDs) | |
| Amino Acid | | | GPI, VOA, VOP (to | |
| Metabolism, | | | the extent subjects | |
| Inborn Errors+ | | | have MDs) | |
| Amino Acid | | | GPI, VOA, VOP (to | |
| Transport | | | the extent subjects | |
| Disorders, | | | have MDs) | |
| Inborn+ | | | GPI, VOA, VOP (to | |
| Amyloidosis, | | | the extent subjects | |
| Familial+ | | | have MDs) | |
| Brain Diseases, | | | GPI, VOA, VOP (to | |
| Metabolic, | | | the extent subjects | |
| Inborn+ | | | have MDs) | |
| Carbohydrate | | | GPI, VOA, VOP (to | |
| Metabolism, | | | the extent subjects | |
| Inborn Errors+ | | | have MDs) | |
| Hyper- | | | GPI, VOA, VOP (to | |
| bilirubinemia, | | | the extent subjects | |
| Hereditary+ | | | have MDs) | |
| Lipid | | | GPI, VOA, VOP (to | |
| Metabolism, | | | the extent subjects | |
| Inborn Errors+ | | | have MDs) | |
| Lysosomal | | | GPI, VOA, VOP (to | |
| Storage | | | the extent subjects | |
| Diseases+ | | | have MDs) | |
| Metal | | | GPI, VOA, VOP (to | |
| Metabolism, | | | the extent subjects | |
| Inborn Errors+ | | | have MDs) | |
| Peroxisomal | | | GPI, VOA, VOP (to | |
| Disorders+ | | | the extent subjects | |
| | | | have MDs) | |
| Porphyrias+ | | | GPI, VOA, VOP (to | |
| | | | the extent subjects | |
| | | | have MDs) | |
| Mitochondrial | | | GPI, VOA, VOP (to | |
| Diseases | | | the extent subjects | |
| | | | have MDs) | |
| Optic Atrophy, | | CNS | | |
| Autosomal | | visual | | |
| Dominant | | prosthesis | | |
| | | @V1 | | |
| Optic Atrophy, | | CNS | | |
| Hereditary, | | visual | | |
| Leber | | prosthesis | | |
| | | @V1 | | |
| Pyruvate | | | GPI, VOA, VOP (to | |
| Carboxylase | | | the extent subjects | |
| Deficiency | | | have MDs) | |
| Disease | | | | |
| Pyruvate | | | GPI, VOA, VOP (to | |
| Dehydrogenase | | | the extent subjects | |
| Complex | | | have MDs) | |
| Deficiency | | | | |
| Disease | | | | |
| Porphyrias | | | | |
| Porphyria, | | | GPI, VOA, VOP (to | |
| Erythropoietic | | | the extent subjects | |
| | | | have MDs) | |

In certain embodiments, methods are provided for treating a mood disorder by applying electrical stimulation, with the therapy signal parameters disclosed herein, to a target tissue or organ. In certain of these embodiments, the mood disorder may be a condition listed in Table 8. Table 8 provides various spinal cord, cortical, sub-cortical, and/or peripheral targets for applying electrical stimulation in the treatment of each condition. Treatment may be carried out by applying electrical stimulation to any of the targets listed, or to a combination thereof. The list of targets is not exhaustive, meaning that there may be one or more additional targets for each condition.

TABLE 8
Mood disorders

| | Spinal | | | |
|---|---|---|---|---|
| | Cord | Cortical | Sub-Cortical | Peripheral |
| Indication | Target | Target | Target | Target |
| Depressive | | Dorsolater | Subgenual | |
| Disorders | | prefrontal | cingulum, | |
| | | cortex, | posterior | |
| | | orbitofrontal | cingulum, | |
| | | cortex, | NAcc, ventral | |
| | | Cg25, | capsule/ventral | |
| | | Posterior | striatum, | |
| | | cingulate | inferior | |
| | | cortex | thalamic | |
| | | | peduncle, | |
| | | | lateral | |
| | | | habenula, | |
| | | | AIC, BST | |
| Major | | Cg25, | Subgenual | |
| depressive | | Posterior | cingulum, | |
| disorder | | cingulate | posterior | |
| (MDD) | | cortex | cingulum, | |
| | | | NAcc, ventral | |
| | | | capsule/ventral | |
| | | | striatum, | |
| | | | inferior | |
| | | | thalamic | |
| | | | peduncle, | |
| | | | lateral | |
| | | | habenula, | |
| | | | AIC, BST | |
| Dysthymia | | Cg25, | Subgenual | |
| | | posterior | cingulum, | |
| | | cingulate | posterior | |
| | | cortex | cingulum, | |
| | | | NAcc, ventral | |
| | | | capsule/ventral | |
| | | | striatum, | |
| | | | inferior | |
| | | | thalamic | |
| | | | peduncle, | |
| | | | lateral | |
| | | | habenula, | |
| | | | AIC, BST | |
| Double | | Cg25, | Subgenual | |
| depression | | posterior | cingulum, | |
| | | cingulate | posterior | |
| | | cortex | cingulum, | |
| | | | NAcc, ventral | |
| | | | capsule/ventral | |
| | | | striatum, | |
| | | | inferior | |
| | | | thalamic | |
| | | | peduncle, | |
| | | | lateral | |
| | | | habenula, | |
| | | | AIC, BST | |
| Depressive | | Cg25, | Subgenual | |
| Disorder Not | | posterior | cingulum, | |
| Otherwise | | cingulate | posterior | |
| Specified | | cortex | cingulum, | |
| (DD-NOS) | | | NAcc, ventral | |
| | | | capsule/ventral | |
| | | | striatum, | |
| | | | inferior | |
| | | | thalamic | |
| | | | peduncle, | |
| | | | lateral | |
| | | | habenula, | |
| | | | AIC, BST | |

In certain embodiments, methods are provided for treating a visceral pain syndromes by applying electrical stimulation, with the therapy signal parameters disclosed herein, to a target tissue or organ. In certain of these embodiments, the visceral pain syndrome may be a condition listed in Table 9. Table 9 provides various spinal cord, cortical, sub-cortical, and/or peripheral targets for applying electrical stimulation in the treatment of each condition. Treatment may be carried out by applying electrical stimulation to any of the targets listed, or to a combination thereof. The list of targets is not exhaustive, meaning that there may be one or more additional targets for each condition.

TABLE 9
Visceral Pain Syndromes

| | Spinal | Cortical | Subcortical | Peripheral |
|---|---|---|---|---|
| Indication | target | target | target | target |
| Cystitis | S2-4 | Insula, | Vc thalamus, | Pudendal |
| | | S1, S2 | posterior | nerve |
| | | | thalamic | |
| | | | nuclei | |
| IBS | T3-9, | Insula, | | R or L Vagus |
| | L1 | S1, S2 | | nerve, |
| | | | | splanchnic |
| | | | | nerves |
| Mesenteric | T3-9, | Insula | | |
| ischemia | | | | |
| Idiopathic | T3-9 | Insula | Vc, DM | Splanchnic |
| abdominal | | | thalamus, | nerve, R or L |
| pain | | | Posterior | vagus nerve |
| | | | thalamic nuc. | |

“Treating” or “treatment” as used herein with regard to a condition may refer to preventing the condition, reducing, or ending symptoms associated with the condition; generating a complete or partial regression of the condition; or some combination thereof. “Preventing” or “prevention” as used herein with regard to a condition may refer to total or partial prevention of the condition or symptoms associated with the condition.

In certain embodiments, electrical stimulation is performed with at least a portion of the therapy signal at a frequency in a frequency range between about 2 Hz and about 100 kHz; between about 1.5 kHz and about 50 KHz; between about 3 kHz and about 20 KHz; between about 3 kHz and about 15 kHz; or between about 5 kHz and about 15 KHz; or at frequencies of about 5 kHz, about 6 kHz, about 7 kHz, about 8 kHz, about 9 kHz, about 10 KHz, about 11 kHz, or about 12 kHz; and in one embodiment, surprisingly effective results have been found when treating certain medical conditions with frequencies between 5 KHz and 15 kHz, and in one embodiment 10 KHz. (The term “about” is intended to represent +/−10%, or a range as would be understood as reasonably equivalent by one of ordinary skill in the art.)

In various embodiments, the electrical stimulation may be applied with at least a portion of the therapy signal at amplitudes within amplitude ranges of: about 0.1 mA to about 20 mA; about 0.5 mA to about 10 mA; about 0.5 mA to about 7 mA; about 0.5 mA to about 5 mA; about 0.5 mA to about 4 mA; about 0.5 mA to about 2.5 mA; and in one embodiment, surprisingly effective results have been found when treating certain medical conditions with amplitudes below 7 mA.

In various embodiments, the electrical stimulation may be applied with at least a portion of the therapy signal having a pulse width within a pulse width range of from about 10 microseconds to about 333 microseconds; from about 10 microseconds to about 166 microseconds; from about 25 microseconds to about 166 microseconds; from about 25 microseconds to about 100 microseconds; from about 30 microseconds to about 100 microseconds; from about 33 microseconds to about 100 microseconds; from about 50 microseconds to about 166 microseconds; and in one embodiment, surprisingly effective results have been found when treating certain medical conditions with pulse widths from about 25 microseconds to about 100 microseconds; and from about 30 microseconds to about 40 microseconds. In a particular embodiment, the therapy signal at a frequency in a frequency range of 1.5 kHz to 100 kHz, a pulse width in a pulse width range of 10 microseconds to 333 microseconds and an amplitude in an amplitude range of 0.1 mA to 20 mA. The therapy signal can be applied at a duty cycle of 5% to 75%, and can be applied to thoracic spinal cord locations to treat back and/or leg pain, e.g., chronic back and/or leg pain. In another particular embodiment, a therapy signal having a pulse width is applied to the spinal cord at a pulse width in a pulse width range of 10 microseconds to 333 microseconds at any of a variety of suitable frequencies (within or outside the range of 1.5 kHz to 100 kHz) to treat a variety of pain indications, including but not limited to chronic low back pain and/or leg pain.

Application of electrical stimulation in conjunction with the methods disclosed herein can be carried out using suitable devices and programming modules specifically programmed to carry out any of the methods described herein. A variety of devices for administering an electrical signal to a target tissue or organ are taught in the references incorporated by reference above. Other examples of devices for administering an electrical signal in conjunction with SCS are disclosed in US Patent Publications Nos. 2010/0274316 (Atty Dkt. No. 662458024US) and 2010/0211135 (Atty Dkt. No. 662458012US2), both of which are incorporated herein by reference in their entireties. In certain embodiments, a device that is used for applying an electrical signal to the spinal cord may be repurposed with or without modifications to administer an electrical signal to another target tissue or organ, e.g., a cortical, sub-cortical, intra-cortical, or peripheral target. Electrical stimulation may be applied directly to a target tissue or organ, or it may be applied in close proximity to the target tissue or organ (i.e., close enough for the target tissue or organ to receive the electrical signal). As such, any of the herein described systems, sub-systems, and/or sub-components serve as means for performing any of the herein described methods.

In certain embodiments, electrical stimulation is applied to a tissue or organ using a device that comprises a lead, wherein the lead in turn comprises an electrode. In these embodiments, administration of electrical stimulation comprises a positioning step (e.g., placing the lead such that an electrode is in proximity to the target tissue or organ) and a stimulation step (e.g., transmitting an electrical signal (i.e., therapy signal) through the electrode).

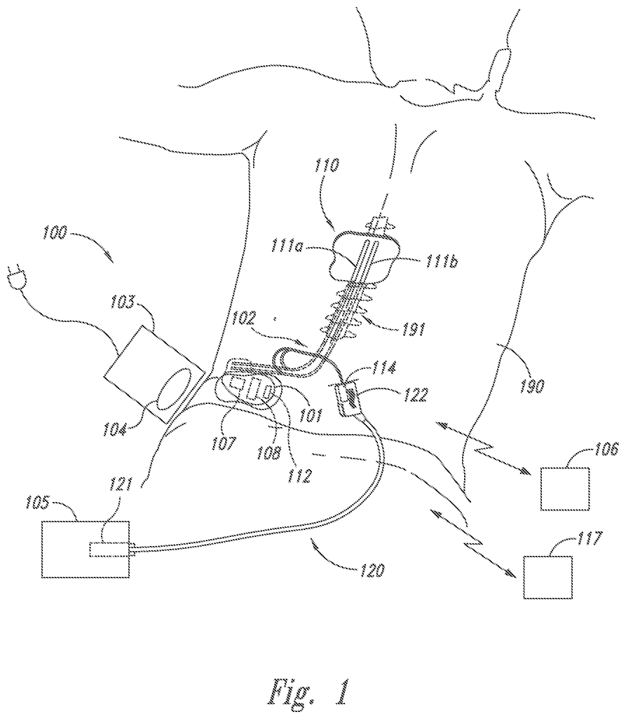

FIG. 1 schematically illustrates a representative treatment system 100 for administering electrical stimulation to the spinal cord 191 in conjunction with the methods disclosed herein. The system 100 can include a pulse generator 101, which may be implanted subcutaneously within a patient 190 and coupled to a signal delivery element 110. In a representative example, the signal delivery element 110 includes one or more leads or lead bodies 111 (shown as first and second leads 111a, 111b) that carry features for delivering therapy to the patient 190 after implantation. The pulse generator 101 can be connected directly to the lead 111, or it can be coupled to the lead 111 via a communication link 102 (e.g., an extension). Accordingly, the lead 111 can include a terminal section that is releasably connected to an extension at a break 114 (shown schematically in FIG. 1). This allows a single type of terminal section to be used with patients of different body types (e.g., different heights). The terms “lead” and “lead body” as used herein include any of a number of suitable substrates and/or support members that carry devices for providing therapy signals to the patient 190. For example, the lead 111 can include one or more electrodes or electrical contacts that direct electrical signals into the patient's tissue. In other embodiments, the signal delivery element 110 can include devices other than a lead body (e.g., a paddle) that also direct electrical signals to the patient 190.

The pulse generator 101 can transmit electrical signals to the signal delivery element 110 that attenuate pathology-induced sodium channel activity and/or modulate GNI. The pulse generator 101 can include a machine-readable (e.g., computer-readable) medium containing instructions for generating and transmitting suitable therapy signals. The pulse generator 101 and/or other elements of the system 100 can include one or more processors 107, memories 108 and/or input/output devices. Accordingly, the process of providing modulation signals and executing other associated functions can be performed by computer-executable instructions contained on computer-readable media, e.g., at the processor(s) 107 and/or memory(s) 108. The pulse generator 101 can include multiple portions, elements, and/or subsystems (e.g., for directing signals in accordance with multiple signal delivery parameters), housed in a single housing, as shown in FIG. 1, or in multiple housings.

The pulse generator 101 can also receive and respond to an input signal received from one or more sources. The input signals can direct or influence the manner in which the therapy instructions are selected, executed, updated and/or otherwise performed. The input signal can be received from one or more sensors 112 (one is shown schematically in FIG. 1 for purposes of illustration) that are carried by the pulse generator 101 and/or distributed outside the pulse generator 101 (e.g., at other patient locations) while still communicating with the pulse generator 101. The sensors 112 can provide inputs that depend on or reflect patient state (e.g., patient position, patient posture and/or patient activity level, or pathophysiology measurements defined as appropriate to the clinical disorder), and/or inputs that are patient-independent (e.g., time). In other embodiments, inputs can be provided by the patient and/or the practitioner, as described in further detail later.

In some embodiments, the pulse generator 101 can obtain power to generate the electrical signals from an external power source 103. The external power source 103 can transmit power to the implanted pulse generator 101 using electromagnetic induction (e.g., radiofrequency (RF) signals). For example, the external power source 103 can include an external coil 104 that communicates with a corresponding internal coil (not shown) within the implantable pulse generator 101. The external power source 103 can be portable for ease of use. In another embodiment, the pulse generator 101 can obtain the power to generate electrical signals from an internal power source, in addition to or in lieu of the external power source 103. For example, the implanted pulse generator 101 can include a non-rechargeable battery or a rechargeable battery to provide such power. When the internal power source includes a rechargeable battery, the external power source 103 can be used to recharge the battery. The external power source 103 can in turn be recharged from a suitable power source (e.g., conventional wall power).

In some cases, an external programmer 105 (e.g., a trial modulator) can be coupled to the signal delivery element 110 during an initial implant procedure, prior to implanting the pulse generator 101. For example, a practitioner (e.g., a physician and/or a company representative) can use the external programmer 105 to vary the modulation parameters provided to the signal delivery element 110 in real time, and select optimal or particularly efficacious parameters. These parameters can include the position of the signal delivery element 110, as well as the characteristics of the electrical signals provided to the signal delivery element 110. In a typical process, the practitioner uses a cable assembly 120 to temporarily connect the external programmer 105 to the signal delivery device 110. The cable assembly 120 can accordingly include a first connector 121 that is releasably connected to the external programmer 105, and a second connector 122 that is releasably connected to the signal delivery element 110. Accordingly, the signal delivery element 110 can include a connection element that allows it to be connected to a signal generator either directly (if it is long enough) or indirectly (if it is not). The practitioner can test the efficacy of the signal delivery element 110 in an initial position. The practitioner can then disconnect the cable assembly 120, reposition the signal delivery element 110, and reapply the electrical modulation. This process can be performed iteratively until the practitioner obtains the desired position for the signal delivery device 110. Optionally, the practitioner may move the partially implanted signal delivery element 110 without disconnecting the cable assembly 120. Further details of suitable cable assembly methods and associated techniques are described in US Patent Publication No. 2011/0071593 (Atty. Dkt. No. 662458022US), which is incorporated herein by reference in its entirety.

After the position of the signal delivery element 110 and appropriate signal delivery parameters are established using the external programmer 105, the patient 190 can receive therapy via signals generated by the external programmer 105, generally for a limited period of time. In a representative application, the patient 190 receives such therapy for one week. During this time, the patient wears the cable assembly 120 and the external programmer 105 outside the body. Assuming the trial therapy is effective or, shows the promise of being effective, the practitioner then replaces the external programmer 105 with the implanted pulse generator 101, and programs the pulse generator 101 with parameters selected based on the experience gained during the trial period. Optionally, the practitioner can also replace the signal delivery element 110. Once the implantable pulse generator 101 has been positioned within the patient 190, the signal delivery parameters provided by the pulse generator 101 can still be updated remotely via a wireless physician's programmer (e.g., a physician's remote) 117 and/or a wireless patient programmer 106 (e.g., a patient remote). Generally, the patient 190 has control over fewer parameters than does the practitioner. For example, the capability of the patient programmer 106 may be limited to starting and/or stopping the pulse generator 101, and/or adjusting the signal amplitude.

In any of the foregoing embodiments, the parameters in accordance with which the pulse generator 101 provides signals can be modulated during portions of the therapy regimen. For example, the frequency, amplitude, pulse width and/or signal delivery location can be modulated in accordance with a preset program, patient and/or physician inputs, and/or in a random or pseudorandom manner. Such parameter variations can be used to address a number of potential clinical situations, including changes in the patient's perception of one or more symptoms associated with the condition being treated, changes in the preferred target neural population, and/or patient accommodation or habituation.

In certain embodiments, electrical stimulation is applied to the dorsal column. In other embodiments, the electrical stimulation is applied to other neural tissue such as nerve roots and peripherals nerves on the spinal level, including for example the dorsal root (DN) and dorsal root ganglion (DRG) and the ventral root (VN). In other embodiments, electrical stimulation may be applied to one or more non-spinal cord tissues or organs. For example, electrical stimulation may be applied to various cortical, sub-cortical, intra-cortical, or peripheral targets. For certain conditions, electrical stimulation may be applied to a single target tissue or organ. For other conditions, electrical stimulation may be applied to multiple target tissues or organs sequentially or simultaneously. For example, where the condition is a chronic pain disorder, stimulation may be applied to the spinal cord, a cortical target, a sub-cortical target, or a combination thereof. In certain embodiments, electrical stimulation parameters are configured so as to not result in the patient experiencing paresthesia.

In certain embodiments, electrical stimulation is applied at an amplitude that is sub-threshold with regard to paresthesia and supra-threshold with regard to symptom reduction (e.g., therapy, such as pain relief). In certain of these embodiments, electrical stimulation is applied at an amplitude between about 0.5 mA to about 20 mA. In certain embodiments, electrical stimulation is applied at a duty cycle. Duty cycles can range from 1% to about 99%, or between about 5% and about 75%, or between about 10% and about 50%.

In certain embodiments of the methods provided herein, electrical stimulation may be administered on a pre-determined schedule. In other embodiments, electrical stimulation may be administered on an as-needed basis. Administration may continue for a pre-determined amount of time, or it may continue indefinitely until a specific therapeutic benchmark is reached, for example until an acceptable reduction in one or more symptoms. In certain embodiments, electrical stimulation may be administered one or more times per day, one or more times per week, once a week, once a month, or once every several months. In certain embodiments, administration frequency may change over the course of treatment. For example, a subject may receive less frequent administrations over the course of treatment as certain therapeutic benchmarks are met. The duration of each administration (e.g., the actual time during which a subject is receiving electrical stimulation) may remain constant throughout the course of treatment, or it may vary depending on factors such as patient health, internal pathophysiological measures, or symptom severity. In certain embodiments, the duration of each administration may range from 1 to 4 hours, 4 to 12 hours, 12 to 24 hours, 1 day to 4 days, or 4 days or greater.

In certain embodiments of the methods provided herein, administration of electrical stimulation may be combined with one or more additional treatment modalities. For example, electrical stimulation may be applied in combination with the administration of one or more pharmaceutical agents that block fast sodium channels. In other embodiments, electrical stimulation may be used as a replacement for other treatment modalities. For example, electrical stimulation may be administered to a subject who has previously received neuroleptics or other sodium channel blockers but who has experienced unsatisfactory results and/or negative side effects. In certain embodiments, application of electrical stimulation may result in a greater treatment effect than administration of other treatment modalities, including for example a larger reduction in symptoms or an increased duration of symptom reduction.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

Example 1. A method of attenuating pathology-induced sodium channel activity comprising applying electrical stimulation to a target neural location, wherein the electrical stimulation includes one or more system parameters as described in the embodiments above, and wherein the target neural location is chosen so as to treat the medical condition listed in Tables 1-9 above.
Example 2. A method of treating a condition associated with increased fast sodium channel comprising applying electrical stimulation to a target neural location, wherein the electrical stimulation includes one or more system parameters as described in the embodiments above, and wherein the target neural location is chosen so as to treat the medical condition listed in Tables 1-9 above.
Example 3. A method of modulating GNI comprising applying electrical stimulation to a target neural location, wherein the electrical stimulation includes one or more system parameters as described in the embodiments above, and wherein the target neural location is chosen so as to treat the medical condition listed in Tables 1-9 above.
Example 4. A neuromodulation system for treating a medical condition comprising: an implantable (or external) pulse generator configured to attenuate pathology-induced sodium channel activity by generating and applying a electrical stimulation to a target neural location, wherein the electrical stimulation includes one or more system parameters as described in the embodiments above, and wherein the target neural location is chosen so as to treat the medical condition listed in Tables 1-9 above.
Example 5. A neuromodulation system for treating a medical condition comprising: an implantable (or external) pulse generator configured to treat a condition associated with increased fast sodium channel by generating and applying a electrical stimulation to a target neural location, wherein the electrical stimulation includes one or more system parameters as described in the embodiments above, and wherein the target neural location is chosen so as to treat the medical condition listed in Tables 1-9 above.
Example 6. A neuromodulation system for treating a medical condition comprising: an implantable (or external) pulse generator configured to modulate GNI by generating and applying a electrical stimulation to a target neural location, wherein the electrical stimulation includes one or more system parameters as described in the embodiments above, and wherein the target neural location is chosen so as to treat the medical condition listed in Tables 1-9 above.

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

1-25. (canceled)

26. A method for treating back or leg pain in a patient by modulating a glial neuronal cell interaction of the patient, the method comprising:
programming a signal generator to deliver multiple electrical therapy signals to the patient's spinal cord via one or more implanted signal delivery elements to treat the patient,
wherein the multiple electrical therapy signals have a frequency of 2 Hz to 100 kHz and modulate a glial neuronal cell interaction of the patient to reduce the back or leg pain in the patient.

27. The method of claim 26 wherein the multiple electrical therapy signals are delivered simultaneously.

28. The method of claim 26 wherein the one or more signal delivery elements include a lead having a plurality of electrical contacts, and wherein the multiple electrical therapy signals are delivered via different electrical contacts of the plurality of electrical contacts.

29. The method of claim 26 wherein the one or more signal delivery elements include multiple leads, and wherein the multiple electrical therapy signal are delivered via different leads.

30. The method of claim 26 wherein the multiple electrical therapy signals are delivered to the patient's spinal cord at a vertebral location between about T8 and T12.

31. The method of claim 26 wherein at least one of the multiple electrical signals does not create paresthesia in the patient.

32. The method of claim 26 wherein at least one of the multiple electrical signals has a pulse width in a pulse width range of from about 10 microseconds to about 333 microseconds.

33. (canceled)

34. The method of claim 26 wherein at least one of the multiple electrical signals has a frequency within a frequency range of from about 40 Hz to about 500 Hz.

35. The method of claim 26 wherein the multiple electrical signals include at least a first electrical signal and a second electrical signal, and wherein the first electrical signal has a different frequency than the second electrical signal.

36. A method for treating back or leg pain in a patient by modulating a glial neuronal cell interaction of the patient, the method comprising:
programming a signal generator to deliver multiple electrical therapy signals to multiple target tissues located in the patient's spinal cord via one or more implanted signal delivery elements to treat the patient,
wherein the electrical therapy signals modulate a glial neuronal cell interaction of the patient to reduce the back or leg so pain in the patient.

37. The method of claim 36 wherein the multiple electrical therapy signals are delivered simultaneously.

38. The method of claim 36 wherein the one or more signal delivery elements include a lead having a plurality of electrical contacts, and wherein the multiple electrical therapy signals are delivered via different electrical contacts of the plurality of electrical contacts.

39. The method of claim 36 wherein the one or more signal delivery elements include multiple leads, and wherein the multiple electrical therapy signal are delivered via different leads.

40. The method of claim 36 wherein the multiple target tissues located in the patient's spinal cord are at a vertebral location between about T8 and T12.

41. The method of claim 36 wherein at least one of the multiple electrical signals does not create paresthesia in the patient.

42. The method of claim 36 wherein at least one of the multiple electrical signals has a pulse width in a pulse width range of from about 10 microseconds to about 333 microseconds.

43. The method of claim 36 wherein the multiple electrical signals include at least a first electrical signal and a second electrical signal, and wherein the first electrical signal has a different frequency than the second electrical signal.

44. A method for treating back or leg pain in pain in a patient by changing an activity of a glial cell of the patient, the method comprising:
programming a signal generator to deliver multiple electrical therapy signals to multiple target tissues located in the patient's spinal cord via one or more implanted signal delivery elements to treat the patient,
wherein the electrical therapy signals produce changes in the activity of the glial cell of the patient to reduce the back or leg pain in the patient.

45. The method of claim 44 wherein the multiple target tissues located in the patient's spinal cord are at a vertebral location between about T8 and T12.

46. The method of claim 44 wherein the multiple electrical signals each have a frequency within a frequency range of from about 2 Hz to about 100 KHz.

47. The method of claim 44 wherein at least one of the multiple electrical signals has a frequency within a frequency range of from about 40 Hz to about 500 Hz.

48. The method of claim 44 wherein the multiple electrical signals include at least a first electrical signal and a second electrical signal, and wherein the first electrical signal has a different frequency than the second electrical signal.

49. The method of claim 44 wherein at least one of the multiple electrical signals does not create paresthesia in the patient.

50. A method for treating back or leg pain in a patient by changing an expression of a glial cell of the patient, the method comprising:
programming a signal generator to deliver multiple electrical therapy signals to multiple target tissues located in the patient's spinal cord via one or more implanted signal delivery elements to treat the patient,
wherein the electrical therapy signals produce changes in the expression of the glial cell of the patient to reduce the back or leg pain in the patient.

51. The method of claim 50 wherein the multiple target tissues located in the patient's spinal cord are at a vertebral location between about T8 and T12.

52. The method of claim 50 wherein the multiple electrical signals each have a frequency within a frequency range of from about 2 Hz to about 100 KHz.

53. The method of claim 50 wherein at least one of the multiple electrical signals has a frequency within a frequency range of from about 40 Hz to about 500 Hz.

54. The method of claim 50 wherein the multiple electrical signals include at least a first electrical signal and a second electrical signal, and wherein the first electrical signal has a different frequency than the second electrical signal.

55. The method of claim 50 wherein at least one of the multiple electrical signals does not create paresthesia in the patient.

56. The method of claim 26 wherein the multiple electrical therapy signals are delivered to multiple target tissues in the patient's spinal cord.
